(19) United States
(12) Patent Application Publication
(10) Pub. No.: US 2013/0171218 A1
(43) Pub. Date: Jul. 4, 2013

(54) DUAL FUNCTION PRODUCT

(71) Applicants: Hy Si Bui, Piscataway, NJ (US); Mohamed Kanji, Edison, NJ (US); Liana Esposito, Franklin Park, NJ (US)

(72) Inventors: Hy Si Bui, Piscataway, NJ (US); Mohamed Kanji, Edison, NJ (US); Liana Esposito, Franklin Park, NJ (US)

(73) Assignee: L'ORÉAL, Paris (FR)

(21) Appl. No.: 13/583,713

(22) PCT Filed: Mar. 10, 2011
(86) PCT No.: PCT/US11/27880

§ 371 Date: Sep. 10, 2012

Related U.S. Application Data
(60) Provisional application No. 61/312,564, filed on Mar. 10, 2010.

(51) Int. Cl. A61K 8/02; A61K 9/06

(52) U.S. Cl. CPC A61K 8/0233; A61K 9/06
USPC 424/401; 424/400; 264/250

(57) ABSTRACT

Disclosed is a dual function product with a receptacle for storing the product; a structured sleeve composition located within the receptacle that has at least one straight-chain low molecular mass N-acyl glutamic acid diamide, at least one N-acyl glutamic acid diamide, at least one gel-promoting solvent, at least one block copolymer having at least one hard segment and at least one soft segment, at least one solvent capable of solubilizing the at least one hard segment and/or the at least one soft segment of the block copolymer, and optionally at least one active ingredient, and a core composition disposed within the sleeve, the core composition having at least one active ingredient; and at least one non-volatile solvent, and wherein the core composition has a melting point less than that of the sleeve composition. Methods of making and using the product are also disclosed.

### CROSS-REFERENCE TO RELATED APPLICATIONS

This application claims the benefit of the filing date of U.S. Provisional Patent Application No. 61/312,564, filed Mar. 10, 2010, the disclosure of which is hereby incorporated herein by reference.

### FIELD OF THE INVENTION

The present invention is directed, in general, to a dual function product. More particularly, the invention is directed to a product comprising at least two different compositions contained in a single receptacle, wherein the compositions provide different functions, possess good storage stability, particularly with respect to variations in temperature, have good payoff and do not require the use of wax as a structuring agent.

### BACKGROUND OF THE INVENTION

Conventional structured compositions typically employ various types of waxes as structuring agents in order to form user-friendly compositions having good pay-off (a term used to describe both the amount of composition applied onto a target substrate, as well as, the way the composition distributes onto the substrate), and stability properties, particularly with respect to temperature stability. The problem with wax-based stick compositions is that they possess an undesirable waxy feel and, if used to impart color, fail to facilitate significant color deposit onto a targeted substrate.

Attempts have been made in the past to formulate structured gel-form compositions in the absence of wax. For example, various types of polyamides have been commercialized as gellators/structuring agents in order to form solid compositions. Similarly, various glutamides, as well as various types of polyurethanes have also been commercialized in order to form solid, preferably clear, compositions. Such attempts, however, while successful at making solid compositions, yielded numerous technical problems.

One of the technical problems associated with the above-referenced, commercial wax-free compositions involves their stability when exposed to elevated temperatures. It is imperative, from a practical point of view, that such compositions be able to withstand fluctuations in temperature during conventional storage conditions without their becoming too soft, thereby negatively impacting their use profile. In order to avoid such stability issues, the composition must possess a certain melting point profile.

Another technical problem relates to the way in which the product is deposited onto a target substrate, also referred to as “pay-off”. Poor pay-off, defined as too much deposit, too little deposit, or lack of uniformity of deposit, is primarily associated with the hardness/elasticity of the structured product. Thus, in order to avoid such deposit issues, it is necessary that the product possess certain hardness/elasticity properties.

It is therefore an object of the present invention to provide a non-aqueous carrier composition capable of forming a gel structure, e.g., a soft gel or a hard or molded gel (such as a gel stick), preferably the latter, and capable of carrying various types of active ingredients that does not suffer from the aforementioned technical problems.

It is yet another object of the present invention to provide a product capable of simultaneously applying at least two different compositions, onto a targeted substrate.

### SUMMARY OF TEE INVENTION

An aspect of the present invention is directed to a dual function product comprising: a) a receptacle for storing the product; b) a structured sleeve composition located within the receptacle, the composition comprising: (i) at least one straight-chain low molecular mass N-acyl glutamic acid diamide; (ii) at least one branched-chain low molecular mass N-acyl glutamic acid diamide; (iii) at least one gel-promoting solvent (iv) at least one high molecular mass block copolymer having at least one hard segment and at least one soft segment; (v) at least one solvent capable of solubilizing the at least one hard segment and/or the at least one soft segment of the block copolymer; and (vi) optionally at least one active ingredient, wherein the structured sleeve composition has a hardness value ranging from about 30 to about 300 gramforce (gf), a melting point of about 50° C. or higher, does not require use of wax as a structuring agent (e.g., is free of wax), and which may be transparent in appearance; and c) a core composition disposed within the sleeve, the core composition comprising: vii) at least one active ingredient; and viii) at least one non-volatile solvent, and wherein the core composition has a melting point less than that of the sleeve composition.

Another aspect of the present invention is directed to a process for making a dual function product involving the steps of: a) providing a receptacle; b) forming a structured sleeve composition by employing the steps of: i) providing a first sleeve composition comprising: (1) at least one straight-chain low molecular mass N-acyl glutamic acid diamide having a straight-chain alkyl group; (2) at least one branched-chain low molecular mass N-acyl glutamic acid diamide having a branched-chain alkyl group; and (3) at least one gel-promoting solvent; ii) providing a second sleeve composition including: (3) at least one high molecular mass block copolymer having at least one hard segment and at least one soft segment; and (4) at least one solvent capable of solubilizing the at least one hard segment and/or the at least one soft segment of the block copolymer; (5) optionally, at least one active ingredient; c) mixing the first sleeve composition and the second sleeve composition at a temperature of from about 90° C. to about 125° C., to form a heated composition; d) pouring the heated composition into the receptacle and allowing it to cool, thereby forming a sleeve within the receptacle, wherein the structured sleeve composition has a hardness value ranging from about 30 to about 300 gf, a melting point of about 50° C. or higher, does not require use of wax as a structuring agent (e.g., is free of wax), and which may be transparent in appearance; e) providing a core composition comprising: i) at least one active ingredient; and ii) at least one non-volatile solvent, wherein the core composition has a melting point less than that of the structured sleeve composition; and f) pouring the core composition into the structured sleeve. Preparing the compositions at these temperatures minimizes both the cost, and degree of manufacturing difficulty.

### BRIEF DESCRIPTION OF THE DRAWING

FIG. 1 is color photograph of one embodiment of the present invention.

### DETAILED DESCRIPTION OF THE INVENTION

Other than in the operating examples, or where otherwise indicated, all numbers expressing quantities of ingredients and/or reaction conditions are to be understood as being modified in all instances by the term “about” which as used herein, refers to ±10-±15% of the expressed value.

“Non-volatile”, as used herein, means having a flash point of greater than about 100° C.

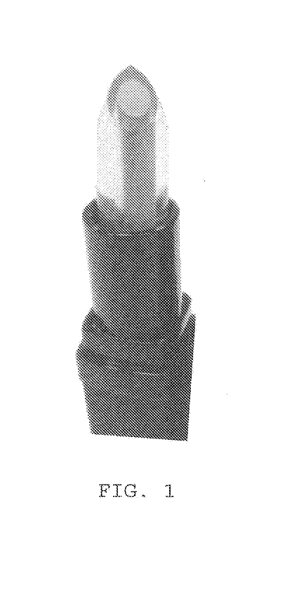

The term “transparent” is defined herein as having the property of transmitting rays of light through its substance so that bodies situated beyond or behind can be distinctly seen, as seen in FIG. 1.

As used herein, “structured” means rigidified.

### Structured Sleeve

The structured sleeve composition of the present invention can be prepared from a combination of a first sleeve composition and a second sleeve composition.

The sleeve composition, in general, comprises a mixture of: at least one branched-chain low molecular mass (wherein the term “low molecular mass” as used herein refers to a molecular mass from greater than zero up to about 2,000 daltons) organogellator; at least one straight-chain low molecular mass organogellator; at least one solvent capable of forming hydrogen bonds with the organogellators; at least one high molecular mass block copolymer; at least one solvent for solubilizing the block copolymer; and at least one active ingredient.

### First Sleeve Composition

### Low Molecular Mass Organogellators

The low molecular mass gelators for use in the present invention are generally chosen from at least one N-acyl glutamic acid diamide having a straight-chain alkyl group, such as dibutyl lauroyl glutamide, and at least one N-acyl glutamic acid diamide having a branched-chain alkyl group, such as dibutyl ethylhexanoyl glutamide.

In a preferred embodiment, the dibutyl lauroyl glutamide is employed in an amount of from about 0.1 to about 50% by weight, such as from about 0.2 to about 40% by weight, and from about 0.3 to about 30% by weight, all weights being based on the total weight of the first sleeve composition.

Similarly, the dibutyl ethylhexanoyl glutamide is employed in an amount of from about 0.1 to about 50% by weight, such as from about 0.2 to about 40% by weight, and from about 0.3 to about 30% by weight, all weights being based on the total weight of the first sleeve composition.

In a preferred embodiment, to obtain a transparent appearance, the dibutyl lauroyl glutamide and dibutyl ethylhexanoyl glutamide are employed, collectively, in an amount of from about 0.1 to about 7% by weight, such as from about 1 to about 5% by weight, and from about 1.5 to about 3% by weight, all weights being based on the total weight of the resultant sleeve composition.

The dibutyl lauroyl glutamide is commercially available as GP-1 and the dibutyl ethylhexanoyl glutamide is commercially available as EB-21, both from Ajinomoto of Fort Lee, N.J.

In a preferred embodiment, the N-acyl glutamic acid diamide having a straight chain alkyl group and N-acyl glutamic acid diamide having a branched chain alkyl group are employed in a ratio by weight of from about 1:1 to about 3:1, and preferably about 1.5:1.

### Gel-Promoting Solvent

The low molecular mass organogellators of the present invention are solubilized in a solvent capable of promoting gel formation. Polar and non-polar solvents may be utilized. Solvents capable of forming hydrogen bonds include, for example, alcohols, monoalcohols, dialcohols, acids, esters, and the like.

It is preferred to utilize a polar solvent for its increased ability to form hydrogen bonds with the organogellators. Preferred polar solvents include, but are not limited to, C2-C5 glycols, such as propylene glycol, butylene glycol and pentylene glycol. These solvents are believed to promote gel formation by inhibiting intercalation (intramolecular bonding) in the glutamide molecules. Other preferred solvents include, for example, octododecanol, isostearyl alcohol, and the like. Yet other preferred solvents include substituted hydrocarbyl siloxanes, as disclosed, for example, in U.S. Patent Application Publication 2004/0223936 A1. They are believed to promote hydrogen bond formation between molecules of the glutamides. One exemplary substituted hydrocarbyl siloxane is CARBINOL FLUID, bis-hydroxyethoxypropyl dimethicone, which is a hydrocarbyl functional organopolysiloxane having the formula, R¹Me₂SiO(Me₂SiO)ₓSiMe₂R¹ where R¹ is —(CH₂)₂OCH₂CH₂OH, and x is such to provide the product with a viscosity of about 50 cS (mm₂/s) at 23° C. The solvents listed herein may be used individually or in combination of two or more.

It is preferred that the solvents be capable of dissolving the organogellators at a temperature of from about 90° C. to about 125° C.

The at least one gel-promoting solvent will typically be employed in an amount of from about 10 to about 99% by weight, such as from about 20 to about 90% by weight, and from about 30 to about 80% by weight, all weights being based on the total weight of the first sleeve composition. In a preferred embodiment, the gel-promoting solvent is employed in an amount of from about 3 to about 50% by weight, such as from about 5 to about 40% by weight, and from about 7 to about 20% by weight, all weights being based on the total weight of the resultant sleeve composition.

### Second Sleeve Composition

The second sleeve composition of the present invention is formed by combining at least one high molecular mass block copolymer (wherein the term “high molecular mass” as used herein refers to a molecular mass of greater than about 5,000 daltons) having at least one hard segment and at least one soft segment with at least one solvent capable of solubilizing the hard and/or soft segments of the block copolymer.

### Block Copolymer

The block copolymers of the present invention are characterized by the presence of at least one “hard” segment, and at least one “soft” segment. Aside from their compositional nature, the hard and soft segments of the block copolymers of the present invention are defined in terms of their respective glass transition temperatures, “T_{g}”. More particularly, the hard segment has a T_{g} of 50° C. or more, whereas the soft segment has a T_{g} of 20° C. or less. The glass transition temperature T_{g} for the hard block can range from 50° C. to 150° C.; 60° C. to 125° C.; 70° C. to 120° C.; 80° C. to 110° C. The glass transition temperature T_{g} for the soft segment of the block copolymer can range from 20° C. to −150° C.; 0° C. to −135° C.; −10° C. to −125° C.; or in some embodiments, −25° C. to −100° C. A more in-depth explanation can be found in U.S. Pat. Nos. 5,294,438 and 6,403,070, the entire contents of which are hereby incorporated by reference.

One type of block copolymer which may be employed by the present invention is a thermoplastic elastomer. The hard segments of the thermoplastic elastomer typically comprise vinyl monomers in varying amounts. Examples of suitable vinyl monomers include, but are not limited to, styrene, methacrylate, acrylate, vinyl ester, vinyl ether, vinyl acetate, and the like.

The soft segments of the thermoplastic elastomer comprise olefin polymers and/or copolymers which may be saturated, unsaturated, or combinations thereof. Suitable olefin copolymers may include, but are not limited to, ethylene/propylene copolymers, ethylene/butylene copolymers, propylene/butylene copolymers, polybutylene, polyisoprene, polymers of hydrogenated butanes and isoprenes, and mixtures thereof.

Thermoplastic elastomers useful in the present invention are block copolymers e.g., di-block, tri-block, multi-block, radial and star block copolymers, and mixtures and blends thereof. A di-block thermoplastic elastomer is usually defined as an A-B type or a hard segment (A) followed by a soft segment (B) in sequence. A tri-block is usually defined as an A-B-A type copolymer or a ratio of one hard, one soft, and one hard segment. Multi-block or radial block or star block thermoplastic elastomers usually contain any combination of hard and soft segments, provided that the elastomers possess both hard and soft characteristics.

In some embodiments, the thermoplastic elastomer of the present invention may be chosen from the class of Kraton™ rubbers (Shell Chemical Company) or from similar thermoplastic elastomers. Kraton™ rubbers are thermoplastic elastomers in which the polymer chains comprise a di-block, tri-block, multi-block or radial or star block configuration or numerous mixtures thereof. The Kraton™ tri-block rubbers have polystyrene (hard) segments on each end of a rubber (soft) segment, while the Kraton™ di-block rubbers have a polystyrene (hard) segment attached to a rubber (soft) segment. The Kraton™ radial or star configuration may be a four-point or other multipoint star made of rubber with a polystyrene segment attached to each end of a rubber segment. The configuration of each of the Kraton™ rubbers forms separate polystyrene and rubber domains.

Each molecule of Kraton™ rubber is said to comprise block segments of styrene monomer units and rubber monomer and/or co-monomer units. The most common structure for the Kraton™ triblock copolymer is the linear A-B-A block type styrene-butadiene-styrene, styrene-isoprene-styrene, styrene-ethylenepropylene-styrene, or styrene-ethylenebutylene-styrene. The Kraton™ di-block is preferably the AB block type such as styrene-ethylenepropylene, styrene-ethylenebutylene, styrene-butadiene, or styrene-isoprene. The Kraton™ rubber configuration is well known in the art and any block copolymer elastomer with a similar configuration is within the practice of the invention. Other block copolymers are sold under the tradename Septon (which represent elastomers known as SEEPS, sold by Kurary, Co., Ltd) and those sold by Exxon Dow under the tradename Vector™.

Other thermoplastic elastomers useful in the present invention include those block copolymer elastomers comprising a styrene-butylene/ethylene-styrene copolymer (tri-block), an ethylene/propylene-styrene copolymer (radial or star block) or a mixture or blend of the two. (Some manufacturers refer to block copolymers as hydrogenated block copolymers, e.g. hydrogenated styrene-butylene/ethylene-styrene copolymer (tri-block)).

The amounts of the block (co)polymer or (co)polymers, as well as their structure (di-block, tri-block, etc.), affect the nature of the thermoplastic elastomer, including its gelled form, which may range from fragile to soft/flexible to firm. For instance, soft gels contain relatively high amounts of soft segments, and firm gels contain relatively high amounts of hard segments. The overall properties of the composition may also be affected by including more than one such block copolymer e.g., including a mixture of copolymers. For example, the presence of tri-block copolymers enhances the integrity of the film formed. The gel may also be transparent, translucent or opaque, depending upon the other cosmetically acceptable ingredients added, as described herein.

It is preferred that the styrene content of the block copolymer be less than 30% by weight, preferably less than 25% by weight, and more preferably less than 20% by weight, based on the weight of the block copolymer. This is because of the tendency of block copolymers having a styrene content of greater than 30% by weight to harden/gel in conventional carrier systems. However, in the event that a block copolymer having a styrene content of greater than 30% by weight is used, it may be necessary to also employ a co-solvent or functional ingredient capable of dissolving the styrene block in an amount effective to control the hardening/gelling of the styrene-containing elastomer in the cosmetic composition.

A particularly preferred block copolymer for use in the present invention is a combination of di-block and tri-block copolymers of styrene-ethylene/butylene-styrene, commercially available from Shell Chemical Company under trade name Kraton G1657M. However, any thermoplastic elastomer of the block copolymer type having at least one soft and at least one hard segment may be used without departing from the spirit of the invention.

The block copolymer is typically employed in an amount of from about 1 to about 40% by weight, such as from about 2 to about 20% by weight, and from about 3 to about 10% by weight, based on the total weight of the second sleeve composition. In a preferred embodiment, the block copolymer is employed in an amount of from about 1 to about 8% by weight, such as from about 2 to about 6% by weight, and from about 3 to about 5% by weight, all weights being based on the total weight of the resultant sleeve composition.

### Solvent for Block Copolymer

Solvents capable of solubilizing the hard and/or soft segment of the block copolymer which may be used herein are typically characterized in terms of their ability to solubilize the hard and/or soft segment at a temperature of from about 100° C. or less.

Nonvolatile solvents capable of solubilizing the hard segment of the block copolymer which can be used in the invention include, but are not limited to, monoesters, diesters, triesters, mixed aliphatic and/or aromatic, polar oils such as: hydrocarbon-based oils of animal origin, such as perhydrosqualene; hydrocarbon-based plant oils such as liquid triglycerides of fatty acids and of glycerol, in which the fatty acids may have varied chain lengths, these chains being linear or branched, and saturated or unsaturated. These oils can be chosen, for example, from wheat germ oil, sunflower oil, corn oil, soybean oil, marrow oil, grapeseed oil, blackcurrant seed oil, sesame oil, hazelnut oil, apricot oil, macadamia oil, castor oil, avocado oil, karite butter, sweet almond oil, cotton oil, alfalfa oil, poppy oil, pumpkin oil, evening primrose oil, millet oil, barley oil, quinoa oil, olive oil, rye oil, safflower oil, candlenut oil, passion flower oil, musk rose oil and caprylic/capric acid triglycerides such as those sold by the company Stearineries Dubois or those sold under the names Miglyol 810, 812 and 818 by the company Dynamit Nobel; natural or synthetic esters of formula R₁COOR₂, wherein R₁ is a higher fatty acid residue having 7 to 19 carbon atoms, and R₂ is a branched hydrocarbon-based chain having 3 to 20 carbon atoms, such as, for example, purcellin oil (cetostearyl octanoate), isopropyl myristate and alkyl or polyalkyl octanoates, decanoates or ricinoleates; synthetic ethers of formula R³COR⁴, wherein R³ is a C₃ to C₁₉ alkyl radical, and R⁴ is a C₃ to C₂₀ alkyl radical; fatty alcohols comprising at least 12 carbon atoms, such as octyldodecanol or oleyl alcohol; cyclic hydrocarbons such as (alkyl)cycloalkanes, wherein the alkyl chain is linear or branched, saturated or unsaturated and has 1 to 30 carbon atoms, such as cyclohexane or dioctylcyclohexane; aromatic hydrocarbons, for example, alkenes such as benzene, toluene, 2,4-dimethyl-3-cyclohexene, dipentene, p-cymene, naphthalene or anthracene, and esters such as isostearyl benzoate; primary, secondary or tertiary amines such as triethanolamine; and mixtures thereof. In one embodiment, synthetic esters such as isopropyl myristate are used.

Preferred esters are those having a weight average molecular weight (Mw) in the range of 100 to 600, preferably from 100 to 500. Examples thereof include, but are not limited to, C12-15 alkyl benzoate, isopropyl myristate (Mw=270), isopropyl palmitate (Mw=300), isononyl isononanoate, cetyl ethylhexanoate (Mw=368), neopentyl glycol diethylhexanoate (Mw=356), diisopropyl sebacate (Mw=286).

The solvent capable of solubilizing the soft segment of the block copolymer may be selected from volatile solvents and nonvolatile solvents. The expression “volatile solvent” means a solvent that is capable of evaporating at room temperature from a support onto which it has been applied, in other words a solvent which has a measurable vapor pressure at room temperature. See, U.S. Pat. No. 6,656,458, the entire content of which is hereby incorporated by reference.

Representative examples of suitable volatile organic solvents include, but are not limited to, volatile hydrocarbon-based oils. The expression “hydrocarbon-based oil” means oil containing only hydrogen and carbon atoms. Examples of volatile hydrocarbon-based oils include isoparaffins, i.e., branched alkanes containing from 8 to 16 carbon atoms, and in particular isododecane (also known as 2,2,4,4,6-pentamethylheptane). It is also possible to use mixtures of such isoparaffins. Other volatile hydrocarbon-based oils, such as petroleum distillates, can also be used.

Suitable nonvolatile solvents which can be used are those having a weight average molecular weight in the range of 150 to 450, preferably from 200 to 350. Examples thereof include, but are not limited to, hydrogenated polydecene, hydrogenated polyisobutene, isoeicosane, polydecene and polybutene.

The solvent capable of solubilizing the hard and/or soft segment of the block copolymer, at a temperature of from 90 to about 125° C., is typically present in an amount of from about 10% to about 99% by weight, such as from about 20 to about 90% by weight, and from about 30 to about 80% by weight, based on the total weight of the second sleeve composition. In a preferred embodiment, the solvent capable of solubilizing the hard and/or soft segment of the block copolymer is employed in an amount of from about 10 to about 50% by weight, such as from about 15 to about 40% by weight, and from about 20 to about 30% by weight, all weights being based on the total weight of the resultant sleeve composition.

### Sleeve Active Ingredients

Various types of active ingredients may be contained in the structured sleeve composition, if desired. Examples of suitable active ingredients include, for example, colorants such as pigments, inks and lakes; dermatological ingredients such as sunscreen agents, anti-acne agents, anti-aging compounds; insect repelling agents; transdermal pharmaceutical compounds; deodorant and antiperspirant agents; perfumes; dye compounds; etc.

The type and amount of active ingredient to be employed will depend on ultimate purpose of the structured sleeve and can be determined by those of ordinary skill in the art.

In order to arrive at a structured sleeve composition having a clear or transparent appearance, the first sleeve composition should employ the low molecular mass organogellators in an amount of less than about 7% by weight, based on the weight of the resultant sleeve composition, and the high molecular mass block copolymer in an amount of less than about 10% by weight, based on the total weight of the resultant sleeve composition. The compositions of the invention, including both sleeve and core compositions alike, that are transparent may contain no colorant or colorant in an amount less than about 0.5% by weight. Compositions that contain colorant and which are colored in appearance will generally contain more than about 0.5% colorant.

Important considerations associated with the structured sleeve composition of the present invention include: reduction in syneresis/storage stability of the product, amount of active ingredient employed in the structured sleeve, and hardness/elasticity/flexibility of the structured sleeve.

These properties are all affected by the weight ratio of low molecular mass organogellators to high molecular mass block copolymer present in the structured sleeve composition. If too much block copolymer is employed relative to the amount of low molecular mass organogellators, the structured sleeve exhibits: less transparency in appearance; increased hardness; decreased elasticity/flexibility; and poor payoff.

The same effect is realized if too much low molecular mass organogellator is employed relative to the amount of block copolymer in the structured sleeve composition.

Thus, the ratio by weight of low molecular mass organogellators to high molecular mass block copolymer needs to be taken into account when making the structured sleeve of the present invention, depending on its final intended use. Suitable ratios by weight of low molecular mass organogellators to high molecular mass block copolymers include from about 1:1 to about 2:1, such as from about 3:1 to about 4:1, and from about 5:1 to about 6:1.

Similarly, the ratio by weight of high molecular mass block copolymer to low molecular mass organogellators needs to be taken into account when making the structured sleeve of the present invention, depending on its final intended use. Suitable ratios by weight of high molecular mass block copolymers to low molecular mass organogellators include from about 1:1 to about 2:1, such as from about 3:1 to about 4:1, and from about 5:1 to about 6:1.

The structured sleeve composition be stable under conventional storage conditions. In order to achieve acceptable storage stability, the composition must have a melting point of about 50° C. or higher, such as 90° C. or higher, and 110° C. or higher.

### Hardness

It is equally as important, however, that the transparent structured sleeve composition have good “pay-off”, i.e., the ability to be elegantly and uniformly deposited onto a targeted substrate. This property is dependent on the hardness of the structured sleeve composition. The hardness of the structured sleeve composition may, for example, be expressed in gramforce (gf). The composition of the present invention may, for example, have a hardness ranging from about 30 gf to about 300 gf, such as from about 50 gf to about 120 gf, and further such as from about 60 gf to about 100 gf.

Hardness is measured in one of two ways. A first test for hardness entails penetrating a probe into the composition and in particular using a texture analyzer (for example TA-XT2i from Rheo) equipped with an ebonite cylinder of height 25 mm and diameter 8 mm. The hardness measurement is carried out at 20° C. at the center of 5 samples of the composition. The cylinder is introduced into each sample of composition at a pre-speed of 2 mm/s and then at a speed of 0.5 mm/s and finally at a post-speed of 2 mm/s, the total displacement being 1 mm. The recorded hardness value is that of the maximum peak observed. The measurement error is ±50 gf.

The second test for hardness is known as the “cheese wire” method, which involves cutting an 8.1 mm or preferably 12.7 mm in diameter stick composition and measuring its hardness at 20° C. using a DFGHS 2 tensile testing machine from Indelco-Chatillon Co. at a speed of 100 mm/minute. The hardness value obtained from this method is expressed in grams as the shear force required to cut a stick under the above conditions. According to this method, the hardness of compositions according to the present invention which may be in stick form may, for example, range from 30 gf to 300 gf, such as from 30 gf to 250 gf, for a sample of 8.1 mm in diameter stick, and further such as from 30 gf to 200 gf, and also further such as from 30 gf to 120 gf for a sample of 12.7 mm in diameter stick.

The hardness of the structured sleeve composition of the present invention is such that the composition is self-supporting and can easily disintegrate to form a satisfactory deposit on a targeted substrate. In addition, this hardness may impart good impact strength to the structured sleeve composition, which may be molded, cast, or extruded, for example, in stick or disk form.

### Core Composition

The core composition of the present invention is typically comprised of at least two main ingredients, namely, at least one active ingredient and at least one non-volatile solvent.

### Core Active Ingredients

Suitable core composition active ingredients include, for example, any colorant (pigment, etc.), any pharmaceutically or cosmetically active agent, or any film forming agent known in the art. For example, a cosmetic makeup composition or a paint composition comprising colorant can provide colorant and/or film forming agent to a substrate (e.g., keratin such as skin, lips), wall, frame, etc., during use to provide the substrate with the desired film and/or color. Dyes are yet another example of a cosmetically active agent for purposes of the present invention. Similarly, a pharmaceutical or cosmetic composition comprising a pharmaceutically active agent can provide such active agent to the patient or consumer upon use.

Acceptable colorants include pigments, nacreous pigments, and pearling agents.

Representative nacreous pigments include white nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride.

Representative pigments include white, colored, inorganic, organic, polymeric, nonpolymeric, coated and uncoated pigments. Representative examples of mineral pigments include titanium dioxide, optionally surface-treated, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium.

Representative liposoluble dyes which may be used according to the present invention include Sudan Red, DC Red 17, DC Green 6, β-carotene, soybean oil, Sudan Brown, DC Yellow 11, DC Violet 2, DC Orange 5, annatto, and quinoline yellow.

Acceptable film forming agents and/or rheological agents are known in the art and include, but are not limited to, those disclosed in U.S. Patent Application Publication No. 2004/0170586, the entire content of which is hereby incorporated by reference.

Non-limiting representative examples of acceptable film forming/rheolgocial agents include silicone resins such as, for example, MQ resins (for example, trimethylsiloxysilicates), Tpropyl silsesquioxanes and MK resins (for example, polymethylsilsesquioxanes), silicone esters such as those disclosed in U.S. Pat. Nos. 6,045,782; 5,334,737; and 4,725,658, the disclosures of which are hereby incorporated by reference, polymers comprising a backbone chosen from vinyl polymers, methacrylic polymers, and acrylic polymers and at least one chain chosen from pendant siloxane groups and pendant fluorochemical groups such as those disclosed in U.S. Pat. Nos. 5,209,924; 4,693,935; 4,981,903; 4,981,902; and 4,972,037; and WO 01/32737, the disclosures of which are hereby incorporated by reference, polymers such as those described in U.S. Pat. No. 5,468,477, the disclosure of which is hereby incorporated by reference (a non-limiting example of such polymers is poly(dimethylsiloxane)-g-poly(isobutyl methacrylate), which is commercially available from 3M Company under the tradename VS 70 IBM).

Suitable examples of acceptable liposoluble polymers include, but are not limited to, polyalkylenes, polyvinylpyrrolidone (PVP) or vinylpyrrolidone (VP) homopolymers or copolymers, copolymers of a C₂ to C₃₀, such as C₃ to C₂₂ alkene, and combinations thereof. As specific examples of VP copolymers which can be used in the invention, mention may be made of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate copolymer.

One type of block copolymer which may be employed in the compositions of the present invention is a thermoplastic elastomer. The hard segments of the thermoplastic elastomer typically comprise vinyl monomers in varying amounts. Examples of suitable vinyl monomers include, but are not limited to, styrene, methacrylate, acrylate, vinyl ester, vinyl ether, vinyl acetate, and the like.

The soft segments of the thermoplastic elastomer typically comprise olefin polymers and/or copolymers which may be saturated, unsaturated, or combinations thereof. Suitable olefin copolymers may include, but are not limited to, ethylene/propylene copolymers, ethylene/butylene copolymers, propylene/butylene copolymers, polybutylene, polyisoprene, polymers of hydrogenated butanes and isoprenes, and mixtures thereof.

Thermoplastic elastomers useful in the present invention include block copolymers e.g., di-block, tri-block, multi-block, radial and star block copolymers, and mixtures and blends thereof. A di-block thermoplastic elastomer is usually defined as an A-B type or a hard segment (A) followed by a soft segment (B) in sequence. A tri-block is usually defined as an A-B-A type copolymer or a ratio of one hard, one soft, and one hard segment. Multi-block or radial block or star block thermoplastic elastomers usually contain any combination of hard and soft segments, provided that the elastomers possess both hard and soft characteristics.

In preferred embodiments, the thermoplastic elastomer of the present invention may be chosen from the class of Kraton™ rubbers (Shell Chemical Company) or from similar thermoplastic elastomers. Kraton™ rubbers are thermoplastic elastomers in which the polymer chains comprise a di-block, tri-block, multi-block or radial or star block configuration or numerous mixtures thereof. The Kraton™ tri-block rubbers have polystyrene (hard) segments on each end of a rubber (soft) segment, while the Kraton™ di-block rubbers have a polystyrene (hard) segment attached to a rubber (soft) segment. The Kraton™ radial or star configuration may be a four-point or other multipoint star made of rubber with a polystyrene segment attached to each end of a rubber segment. The configuration of each of the Kraton™ rubbers forms separate polystyrene and rubber domains.

Each molecule of Kraton™ rubber is said to comprise block segments of styrene monomer units and rubber monomer and/or co-monomer units. The most common structure for the Kraton™ triblock copolymer is the linear A-B-A block type styrene-butadiene-styrene, styrene-isoprene-styrene, styrene-ethylenepropylene-styrene, or styrene-ethylenebutylene-styrene. The Kraton™ di-block is preferably the AB block type such as styrene-ethylenepropylene, styrene-ethylenebutylene, styrene-butadiene, or styrene-isoprene. The Kraton™ rubber configuration is well known in the art and any block copolymer elastomer with a similar configuration is within the practice of the invention. Other block copolymers are sold under the tradename Septon (which represent elastomers known as SEEPS, sold by Kurary, Co., Ltd) and those sold by Exxon Dow under the tradename Vector™.

Other thermoplastic elastomers useful in the present invention include those block copolymer elastomers comprising a styrene-butylene/ethylene-styrene copolymer (tri-block), an ethylene/propylene-styrene copolymer (radial or star block) or a mixture or blend of the two. (Some manufacturers refer to block copolymers as hydrogenated block copolymers, e.g. hydrogenated styrene-butylene/ethylene-styrene copolymer (tri-block)).

Acceptable film forming/rheological agents also include water soluble polymers such as, for example, high molecular weight crosslinked homopolymers of acrylic acid, and Acrylates/C10-30 Alkyl Acrylate Crosspolymer, such as the Carbopol® and Pemulen®; anionic acrylate polymers such as Salcare® AST and cationic acrylate polymers such as Salcare® SC96; acrylamidopropylttrimonium chloride/acrylamide; hydroxyethyl methacrylate polymers, Steareth-10 Allyl Ether/Acrylate Copolymer; Acrylates/Beheneth-25 Metacrylate Copolymer, known as Aculyn® 28; glyceryl polymethacrylate, Acrylates/Steareth-20 Methacrylate Copolymer; bentonite; gums such as alginates, carageenans, gum acacia, gum arabic, gum ghatti, gum karaya, gum tragacanth, guar gum; guar hydroxypropyltrimonium chloride, xanthan gum or gellan gum; cellulose derivatives such as sodium carboxymethyl cellulose, hydroxyethyl cellulose, hydroxymethyl carboxyethyl cellulose, hydroxymethyl carboxypropyl cellulose, ethyl cellulose, sulfated cellulose, hydroxypropyl cellulose, methyl cellulose, hydroxypropylmethyl cellulose, microcrystalline cellulose; agar; pectin; gelatin; starch and its derivatives; chitosan and its derivatives such as hydroxyethyl chitosan; polyvinyl alcohol, PVM/MA copolymer, PVM/MA decadiene crosspolymer, poly(ethylene oxide) based thickeners, sodium carbomer, and mixtures thereof.

The core active ingredient may typically be employed in an amount of from about 0.01 to about 20% by weight, such as from about 0.1 to about 10% by weight, and all subranges therebetween, all weights being based on the weight of the core composition.

### Core Solvent

Suitable solvents for the core active ingredient include volatile and/or non-volatile oils. Such oils can be any acceptable oil including but not limited to silicone oils and/or hydrocarbon oils.

According to preferred embodiments, the solvent comprises one or more volatile silicone oils. Examples of such volatile silicone oils include linear or cyclic silicone oils having a viscosity at room temperature less than or equal to 6 cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE 1

| | Flash | |
|---|---|---|
| | Point | Viscosity |
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane(L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |

Further, a volatile silicone oil that is linear may be employed in the present invention. Suitable volatile linear silicone oils include those described in U.S. Pat. No. 6,338,839 and WO03/042221, the contents of which are incorporated herein by reference. In one embodiment the volatile linear silicone oil is decamethyltetrasiloxane. In another embodiment, the decamethyltetrasiloxane is further combined with another solvent that is more volatile than decamethyltetrasiloxane.

According to other preferred embodiments, the solvent comprises one or more non-silicone volatile oils selected from volatile hydrocarbon oils, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isododecane, isodecane, and for example, the oils sold under the trade names of Isopar or Permethyl.

Non-limiting examples of volatile non-silicone volatile oils are given in Table 2 below.

TABLE 2

| | Flash |
|---|---|
| Compound | Point (° C.) |
| Isododecane | 43 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |

Examples of non-volatile oils that may be used in the present invention include, but are not limited to, polar oils such as:

a.—hydrocarbon-based plant oils with a high triglyceride content consisting of fatty acid esters of glycerol, the fatty acids of which may have varied chain lengths, these chains possibly being linear or branched, and saturated or unsaturated; these oils are especially wheat germ oil, corn oil, sunflower oil, karite butter, castor oil, sweet almond oil, macadamia oil, apricot oil, soybean oil, rapeseed oil, cottonseed oil, alfalfa oil, poppy oil, pumpkin oil, sesame seed oil, marrow oil, avocado oil, hazelnut oil, grape seed oil, blackcurrant seed oil, evening primrose oil, millet oil, barley oil, quinoa oil, olive oil, rye oil, safflower oil, candlenut oil, passion flower oil or musk rose oil; or caprylic/capric acid triglycerides, for instance those sold by the company Stearineries Dubois or those sold under the names Miglyol 810, 812 and 818 by the company Dynamit Nobel;
b.—synthetic oils or esters of formula R₅COOR₆ in which R₅ represents a linear or branched higher fatty acid residue containing from 1 to 40 carbon atoms, including from 7 to 19 carbon atoms, and R₆ represents a branched hydrocarbon-based chain containing from 1 to 40 carbon atoms, including from 3 to 20 carbon atoms, such as, for example, Purcellin oil (cetostearyl octanoate), isononyl isononanoate, C₁₂ to C₁₅ alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; and pentaerythritol esters;
c.—synthetic ethers containing from 10 to 40 carbon atoms;
d.—C₈ to C₂₆ fatty alcohols, for instance oleyl alcohol; and
e.—mixtures thereof.

Examples of non-volatile oils that may be used in the present invention include, but are not limited to, non-polar oils such as branched and unbranched hydrocarbons and hydrocarbon waxes including polyolefins, in particular Vaseline (petrolatum), paraffin oil, squalane, squalene, hydrogenated polyisobutene, hydrogenated polydecene, polybutene, mineral oil, pentahydrosqualene, and mixtures thereof.

The solvent may be employed in an amount of from about 10 to about 90% by weight, such as from about 20 to about 80% by weight, and all subranges therebetween, all weights based on the total weight of the core composition.

The most important feature of the core composition is that it possesses a melting point which is less than that of the outer sleeve composition.

The core composition and the sleeve composition can be prepared according to known processes commonly used in the formulation industry.

Generally, the sleeve composition is hot cast inside a mould comprising a removable central core with the diameter desired for the core composition. After cooling, the central core of the mould is removed, via the end of the mould opposite that defining the surface of application, generally bevelled, of the stick, thus giving way to a tube into which a hollow needle is introduced. The core composition, after optional heating for the purpose of lowering the viscosity thereof, is then introduced by means of the hollow needle into the tube until the latter is filled. After cooling, the stick is removed from the mould and subsequently packaged in a conventional fashion.

The present invention will be better understood from the examples which follow, all of which are intended for illustrative purposes only, and are not meant to unduly limit the scope of the invention in any way.

### Example 1

| Sleeve Composition | % |
|---|---|
| HYDROGENATED STYRENE/BUTADIENE COPOLYMER | 3 |
| HYDROGENATED STYRENE/METHYL | 9 |
| STYRENE/INDENE COPOLYMER | |
| HYDROGENATED POLYDECENE | 15 |
| ISOEICOSANE | 12 |
| NEOPENTYL GLYCOL DICAPRATE | 10 |
| STEARYL HEPTANOATE | 5 |
| BIS-BEHENYL/ISOSTEARYL/PHYTOSTERYL DIMER | 5 |
| DILINOLEYL DIMER DILINOLEATE | |
| SUCROSE ACETATE ISOBUTYRATE | 10 |
| TRICAPRYLIN | 11 |
| OCTYLDODECANOL | 15 |
| DIBUTYL LAUROYL GLUTAMIDE | 3 |
| DIBUTYL ETHYLHEXANOYL GLUTAMIDE | 2 |

### Procedure for Sleeve Composition:

1. Heated HYDROGENATED POLYDECENE with HYDROGENATED STYRENE/BUTADIENE COPOLYMER to 125° C. while mixing.
2. Added HYDROGENATED STYRENE/METHYL STYRENE/INDENE COPOLYMER while mixing until homogenous.
3. Added ISOEICOSANE, NEOPENTYL GLYCOL DICAPRATE, SUCROSE ACETATE ISOBUTYRATE, BUI-BEHNYL/ISOSTEARYL/PHYTOSTEARYL DIMER DILINOLEYL DIMER DILINOLEATE, STEARYL HEPTANOATE, and TRICAPRYLIN while mixing.
4. In a side phase heated OCTYLDODECANOL, DIBUTYL ETHYL GLUTAMIDE and DIBUTYL LAUROYL GLUTAMIDE to 125° C. while mixing.
5. When both phases became homogenous, they were combined and mixed.
6. Poured the hot solution into the outer part of a core mold.
7. After the gel set, placed the mold in the freezer for 30 minutes.
8. Then, the mold for the inner formula was prepared.

| Core Composition | % |
|---|---|
| OCTYLDODECANOL | QS |
| TRIDECYL TRIMELLITATE | 13 |
| ETHYLHEXYL PALMITATE | 5 |
| BIS-DIGLYCERYL POLYACYLADIPATE-2 | 10 |
| POLYBUTENE | 20 |
| DISTEARDIMONIUM HECTORITE | 0.6 |
| PROPYLENE CARBONATE | 0.06 |
| POLYETHYLENE | 8.25 |
| MICROCRYSTALLINE WAX | 3 |
| COLORANTS | 0-15 |

### Procedure for Core Composition:

1. In a side phase, prepared some of the OCTYLDODECANOL and heated to 95° C.
2. While homogenizing, added the DISTEARDIMONIUM HECTORITE and allowed to disperse completely.
3. Continued to homogenize the mixture, slowly added PROPYLENE CARBONATE.
4. Homogenized for 30 minutes or until mixture became homogenous and a gel was formed.
5. In the main kettle under a propeller, heated the remaining OCTYLDODECANOL, TRIDECYL TRIMELLITATE, ETHYLHEXYL PALMITATE and POLYBUTENE to 95° C. and mixed until homogenous.
6. Added the side phase of OCTYLDODECANOL, DISTEARDIMONIUM HECTORITE and PROPYLENE CARBONATE.
7. Mixed until homogenous.
8. At 95° C. under a propeller mixer, added POLYETHYLENE and MICROCRYSTALLINE WAX to the main kettle or finished color ground and mixed until homogenous.
9. Added colorants if desired and mixed until homogenous.
10. Poured the hot solution into the inner part of the core mold.
11. Placed the mold in the freezer for 30 minutes.
12. Removed the core sticks.

What is claimed is:

1. A dual function product comprising:
a) a receptacle for storing the product;
b) a structured sleeve composition located within the receptacle, the composition comprising:
(i) at least one straight-chain low molecular mass N-acyl glutamic acid diamide;
(ii) at least one branched-chain low molecular mass N-acyl glutamic acid diamide;
(iii) at least one gel-promoting solvent;
(iv) at least one high molecular mass block copolymer having at least one hard segment and at least one soft segment;
(v) at least one solvent capable of solubilizing the at least one hard segment and/or the at least one soft segment of the block copolymer; and
(vi) optionally, at least one active ingredient, wherein the structured sleeve composition has a hardness value ranging from about 30 to about 300 gf, and a melting point of about 50° C. or higher, does not require use of wax as a structuring agent, and
c) a core composition disposed within the sleeve, the core composition comprising:
i) at least one active ingredient; and
ii) at least one non-volatile solvent, and wherein the core composition has a melting point less than that of the sleeve composition.

2. The product of claim 1 wherein (b)(i) is dibutyl lauroyl glutamide.

3. The product of claim 1 wherein (b)(ii) is dibutyl ethylhexanoyl glut amide.

4. The product of claim 1 wherein (b)(i) and (b)(ii) are employed in a total amount of from about 0.1 to about 7% by weight, based on the weight of the structured sleeve composition.

5. The product of claim 1 wherein (b)(iii) is isostearyl alcohol.

6. The product of claim 1 wherein (b)(iii) is employed in an amount of from about 3 to about 50% by weight, based on the weight of the sleeve composition.

7. The product of claim 1 wherein (b)(iv) is a mixture of diblock and triblock copolymers.

8. The product of claim 1 wherein (b)(iv) is present in an amount of from about 1 to about 8% by weight, based on the weight of the resultant sleeve composition.

9. The product of claim 1 wherein (b)(v) is hydrogenated polydecene.

10. The product of claim 1 wherein (b)(v) is employed in an amount of from about 10 to about 50% by weight, based on the weight of the resultant sleeve composition.

11. The composition of claim 1, wherein the core composition comprises a colorant.

12. The composition of claim 1, wherein the core composition comprises a film-forming polymer.

13. A process for making a dual function product involving the steps of:
a) providing a receptacle;
b) forming a structured sleeve composition by employing the steps of:
i) providing a first sleeve composition comprising:
(1) at least one straight-chain low molecular mass N-acyl glutamic acid diamide having a straight-chain alkyl group;
(2) at least one branched-chain low molecular mass N-acyl glutamic acid diamide having a branched-chain alkyl group; and
(3) at least one gel-promoting solvent;
ii) providing a second sleeve composition comprising:
(4) at least one high molecular mass block copolymer having at least one hard segment and at least one soft segment; and
(5) at least one solvent capable of solubilizing the at least one hard segment and/or the at least one soft segment of the block copolymer; and
(6) optionally, at least one active ingredient;
c) mixing the first sleeve composition and the second sleeve composition at a temperature of from about 90° C. to about 125° C., to form a heated composition;
d) pouring the heated composition into the receptacle and allowing it to cool, thereby forming a structured sleeve within the receptacle, wherein the structured sleeve composition has a hardness value ranging from about 30 to about 300 gf, a melting point of about 50° C. or higher, and does not require use of wax as a structuring agent;
e) providing a core composition comprising:
i) at least one active ingredient; and
ii) at least one non-volatile solvent, wherein the core composition has a melting point less than that of the structured sleeve composition; and
f) pouring the core composition into the structured sleeve.

14. The process of claim 13 wherein (b)(i)(1) is dibutyl lauroyl glutamide.

15. The process of claim 13 wherein (b)(i)(2) is dibutyl ethylhexanoyl glut amide.

16. The process of claim 13 wherein (b)(i)(1) and (b)(i)(2) are employed in a total amount of from about 0.1 to about 7% by weight, based on the weight of the structured sleeve composition.

17. The process of claim 13 wherein (b)(i)(3) is isostearyl alcohol.

18. The process of claim 14 wherein (b)(i)(3) is employed in an amount of from about 3 to about 50% by weight, based on the weight of the structured sleeve composition.

19. The process of claim 13 wherein (b)(ii)(4) is a mixture of diblock and triblock copolymers.

20. The process of claim 13 wherein (b)(ii)(4) is employed in an amount of from about 1 to about 8% by weight, based on the weight of the structured sleeve composition.

21. The process of claim 13 wherein (b)(ii)(5) is hydrogenated polydecene.

22. The process of claim 13 wherein (b)(ii)(5) is employed in an amount of from about 10 to about 50% by weight, based on the weight of the structured sleeve composition.
